(19) United States
(12) Patent Application Publication
(10) Pub. No.: US 2024/0217903 A1
(43) Pub. Date: Jul. 4, 2024

(54) DEACETYLATION PROCESS, COMPOSITIONS, AND USES THEREOF

(71) Applicant: Firmenich SA, Satigny (CH)

(72) Inventors: Eric FREROT, Satigny (CH); Alexandre Philippe NELL, Satigny (CH); Robert WAGNER, Satigny (CH)

(73) Assignee: Firmenich SA, Satigny (CH)

(21) Appl. No.: 18/558,331

(22) PCT Filed: May 25, 2022
(86) PCT No.: PCT/EP2022/064274

§ 371 Date: Oct. 31, 2023

Related U.S. Application Data
(60) Provisional application No. 63/195,932, filed on Jun. 2, 2021.

(30) Foreign Application Priority Data
Jul. 15, 2021 (EP) 21185866.7

(51) Int. Cl. C07C 29/10; C07C 29/84

(52) U.S. Cl. CPC C07C 29/10; C07C 29/84

(57) ABSTRACT

The disclosure relates to a process for deacetylating certain acetyl-containing compounds, as well as the compositions formed from the process. In some embodiments, the acetyl-containing compounds are acetyl-containing compounds derived from avocado. In some aspects, the disclosure also provides uses of the deacetylated compositions to enhance salty taste, umami taste, or kokumi taste. In some embodiments, the disclosure provides ingestible compositions that include the deacetylated compositions and one or more of a salty tastant, an umami tastant, or a kokumi tastant by incorporating such deacetylated compositions into an ingestible composition. In some other aspects, the disclosure provides methods of reducing or eliminating the amount of one or more of a salty tastant, an umami tastant, or a kokumi tastant. In some other aspects, the disclosure provides uses of the deacetylated compositions to mask a bitter taste.

### TECHNICAL FIELD

The disclosure relates to a process for deacetylating certain acetyl-containing compounds, as well as the compositions formed from the process. In some embodiments, the acetyl-containing compounds are acetyl-containing compounds derived from avocado. In some aspects, the disclosure also provides uses of the deacetylated compositions to enhance salty taste, umami taste, or kokumi taste. In some embodiments, the disclosure provides ingestible compositions that include the deacetylated compositions and one or more of a salty tastant, an umami tastant, or a kokumi tastant. In some other aspects, the disclosure provides methods of reducing or eliminating the amount of one or more of a salty tastant, an umami tastant, or a kokumi tastant by incorporating such deacetylated compositions into an ingestible composition. In some other aspects, the disclosure provides uses of the deacetylated compositions to mask a bitter taste.

### DESCRIPTION OF RELATED ART

The taste system provides sensory information about the chemical composition of the external world. Taste transduction is one of the more sophisticated forms of chemically triggered sensation in animals. Signaling of taste is found throughout the animal kingdom, from simple metazoans to the most complex of vertebrates. Mammals are believed to have five basic taste modalities: sweet, bitter, sour, salty, and umami.

Salty, umami tastes as well as kokumi sensation (roundness, mouthfeel) are most commonly associated with the savory tastes, such as that found in meat products, cheeses, tomatoes, mushrooms, soy sauce, fish sauce, miso, fermented products, and the like. Mammals generally perceive such tastes to be a pleasurable sensation. Many of these typical sources of these tastes are high in salt (sodium chloride), glutamates, or other compounds believed to be unhealthy if ingested in high quantities.

For example, excessive sodium intake can cause a number of health-related problems. One such problem is hypertension. Hypertension is a condition in which the pressure of the blood against artery walls is high enough that it may eventually cause heart disease and other health problems. Excessive sodium intake also adversely affect the balance of water and minerals in the body. For example, excessive sodium intake can cause calcium loss, which can lead to osteoporosis and other problems. Excessive consumption of food products containing glutamates can also have certain adverse health effects, as glutamates may interfere with the functioning of neurotransmitters. Thus, it is generally desirable to reduce the consumption of salt and glutamates. Further, there is increased consumer demand for food and beverage products that do not contain animal-derived ingredients, such as animal-derived fats, meat products, or dairy products, which also impart savory taste.

Enhancement of salt, umami, or kokumi flavors provides an alternative approach to partially or completely replacing ingredients that are traditionally used to impart such tastes.

Even so, there are few compounds, especially naturally derived compounds, that can accomplish this effectively. Thus, there is a continuing need to discover new compounds having utility as flavor modifiers, especially compounds that enhance, among other flavors, the taste of salt, umami, kokumi, or any combination thereof.

### SUMMARY

The present disclosure relates to a deacetylation process that produces, among other things, natural avocado triols that can be used effectively to enhance the taste of salt, umami, kokumi, or any combination thereof, or, in certain instances, to mask a bitter taste.

In a first aspect, the disclosure provides a process for deacetylating avocado acetates, the process comprising:
(a) providing a starting composition comprising one or more avocado acetylate compounds of formula (I)

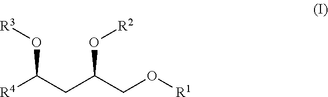

wherein R¹, R², and R³ are independently a hydrogen atom or —C(O)—CH₃, and at least one of R¹, R², and R³ is —C(O)—CH₃, and R⁴ is C₁₃₋₁₇ alkyl, C₁₃₋₁₇ alkenyl, or C₁₃₋₁₇ alkynyl; and
(b) introducing water, an organic acid, and an organic alcohol to the starting composition to hydrolyze the one or more avocado acetate compounds and form a product composition comprising one or more triol compounds of formula (II)

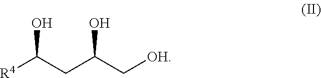

In a second aspect, the disclosure provides a flavor-modifying composition, which is formed by the process of the first aspect, or any embodiments thereof. In a related aspect, the disclosure provides flavor-modifying compositions comprising (2R,4R)-1,2,4-trihydroxy-heptadec-16-ene. In some embodiments thereof, the flavor-modifying composition further comprises (2R,4R)-1,2,4-trihydroxyheptadec-16-yne.

In a third aspect, the disclosure provides uses of the flavor-modifying composition of the second aspect, or any related aspects. In a related aspect, the disclosure provides corresponding methods of using the flavor-modifying compounds of the second aspect, or any related aspects, the method comprising introducing the flavor-modifying composition to an ingestible composition, such as a flavored food or beverage product.

In a fourth aspect, the disclosure provides uses of the flavor-modifying composition of the second aspect, or any related aspects, to modify a flavor of an ingestible composition.

In a related aspect, the disclosure provides corresponding methods of using the flavor-modifying composition of the second aspect, or any related aspects, to modify the flavor of an ingestible composition, the method comprising introducing the flavor-modifying composition to an ingestible composition. In some embodiments, the ingestible composition is a flavored product such as a flavored food or beverage product.

In a fifth aspect, the disclosure provides uses of the flavor-modifying composition of the second aspect, or any related aspects, to enhance a salty taste of an ingestible composition. In related aspects, the disclosure provides uses of the flavor-modifying composition of the second aspect, or any related aspects, to reduce the salt (e.g., sodium chloride) content of an ingestible composition. In a related aspect, the disclosure provides corresponding methods of using any flavor-modifying compositions of the second aspect, or any related aspects, to enhance a salty taste of an ingestible composition, the method comprising introducing the flavor modifying compositions to an ingestible composition. In some embodiments of these aspects, the ingestible composition comprises a salty tastant, such as sodium chloride, potassium chloride, or any combination thereof. In some embodiments, the ingestible composition is a flavored product such as a flavored food or beverage product.

In a sixth aspect, the disclosure provides uses of the flavor-modifying composition of the second aspect, or any related aspects, to enhance an umami taste of an ingestible composition. In related aspects, the disclosure provides uses of the flavor-modifying composition of the second aspect, or any related aspects, to reduce or eliminate the glutamate or aspartate content of an ingestible composition. In related aspects, the disclosure provides corresponding methods of using the flavor-modifying composition of the second aspect, or any related aspects, to enhance an umami taste of an ingestible composition, the method comprising introducing the flavor-modifying composition to an ingestible composition. In some embodiments of these aspects, the ingestible composition is free or substantially free (for example, no more than 1000 ppm) of monosodium glutamate (MSG). In some embodiments, the ingestible composition is a flavored product such as a flavored food or beverage product.

In a seventh aspect, the disclosure provides uses of the flavor-modifying compositions of the second aspect, or any related aspects, to enhance a kokumi taste of an ingestible composition. In a related aspect, the disclosure provides uses of the flavor-modifying compositions of the second aspect, or any related aspects, to reduce or eliminate glutamyl (e.g., L-glutamyl peptides) content of an ingestible composition. In another related aspect, the disclosure provides uses of the flavor-modifying compositions of the second aspect, or any related aspects, to reduce or eliminate animal (e.g., animal broth or meat) content of an ingestible composition. In a related aspect, the disclosure provides corresponding methods of using the flavor-modifying composition of the second aspect, or any related aspects, to enhance a kokumi taste of an ingestible composition, the method comprising introducing the flavor-modifying composition to an ingestible composition. In some embodiments, the ingestible composition is a flavored product such as a flavored food or beverage product.

In an eighth aspect, the disclosure provides uses of the flavor-modifying compositions of the second aspect, or any related aspects, to mask a bitter taste of an ingestible composition. In a related aspect, the disclosure provides corresponding methods of using the flavor-modifying composition of the second aspect, or any related aspects, to mask a bitter taste of an ingestible composition, the method comprising introducing the flavor-modifying composition to an ingestible composition. In some embodiments, the ingestible composition is a flavored product such as a flavored food or beverage product.

In a ninth aspect, the disclosure provides uses of the flavor-modifying compositions of the second aspect, or any related aspects, to enhance a perceived sweetness of an ingestible composition. In a related aspect, the disclosure provides corresponding methods of using the flavor-modifying composition of the second aspect, or any related aspects, to enhance a perceived sweetness of an ingestible composition, the method comprising introducing the flavor-modifying composition to an ingestible composition. In some embodiments, the ingestible composition is a flavored product such as a flavored food or beverage product.

In a tenth aspect, the disclosure provides uses of the flavor-modifying compositions of the second aspect, or any related aspects, to enhance a perceived juiciness of an ingestible composition. In a related aspect, the disclosure provides corresponding methods of using the flavor-modifying composition of the second aspect, or any related aspects, to enhance a perceived juiciness of an ingestible composition, the method comprising introducing the flavor-modifying composition to an ingestible composition. In some embodiments, the ingestible composition is a flavored product such as a flavored food or beverage product.

In an eleventh aspect, the disclosure provides uses of the flavor-modifying compositions of the second aspect, or any related aspects, to enhance a perceived fat content of an ingestible composition. In a related aspect, the disclosure provides corresponding methods of using the flavor-modifying composition of the second aspect, or any related aspects, to enhance a perceived fat content of an ingestible composition, the method comprising introducing the flavor-modifying composition to an ingestible composition. In some related aspects, the disclosure provides uses of the flavor-modifying composition of the second aspect, or related aspects, to reduce the fat content of an ingestible composition. In a related aspect, the disclosure provides methods of reducing the fat content of an ingestible composition, the method comprising introducing the flavor-modifying composition of the second aspect to an ingestible composition. In some embodiments, the ingestible composition is a flavored product such as a flavored food or beverage product.

In a twelfth aspect, the disclosure provides uses of the flavor-modifying compositions of the second aspect, or any related aspects, to enhance a perceived animal product content of an ingestible composition. In a related aspect, the disclosure provides corresponding methods of using the flavor-modifying composition of the second aspect, or any related aspects, to enhance a perceived animal product content of an ingestible composition, the method comprising introducing the flavor-modifying composition to an ingestible composition. In some related aspects, the disclosure provides uses of the flavor-modifying composition of the second aspect, or related aspects, to reduce the animal product content of an ingestible composition. In a related aspect, the disclosure provides methods of reducing the animal product content of an ingestible composition, the method comprising introducing the flavor-modifying composition of the second aspect to an ingestible composition. In some embodiments, the ingestible composition is a flavored product such as a flavored food or beverage product.

In a thirteenth aspect, the disclosure provides uses of the flavor-modifying compositions of the second aspect, or any related aspects, to enhance a perceived alcohol content of an ingestible composition. In a related aspect, the disclosure provides corresponding methods of using the flavor-modifying composition of the second aspect, or any related aspects, to enhance a perceived alcohol content of an ingestible composition, the method comprising introducing the flavor-modifying composition to an ingestible composition. In some embodiments, the ingestible composition is a flavored product such as a flavored food or beverage product. In some embodiments, the ingestible content is a low-alcohol or zero-alcohol beverage.

In a fourteenth aspect, the disclosure provides uses of the flavor-modifying compositions of the second aspect, or any related aspects, to enhance mouthfeel of an ingestible composition. In a related aspect, the disclosure provides corresponding methods of using the flavor-modifying composition of the second aspect, or any related aspects, to enhance mouthfeel an ingestible composition, the method comprising introducing the flavor-modifying composition to an ingestible composition. In some embodiments, the ingestible composition is a flavored product such as a flavored food or beverage product.

In a fifteenth aspect, the disclosure provides uses of the flavor-modifying compositions of the second aspect, or any related aspects, to reduce the perception of an off note of an ingestible composition. In a related aspect, the disclosure provides corresponding methods of using the flavor-modifying composition of the second aspect, or any related aspects, to reduce a perception of an off note an ingestible composition, the method comprising introducing the flavor-modifying composition to an ingestible composition. In some embodiments, the ingestible composition is a flavored product such as a flavored food or beverage product. In some embodiments, the off note is a note characteristic of plant proteins, such as pea proteins, including a cereal note, a cardboard note, a green note, a pungent note, an astringent note, or any combination thereof.

In a sixteenth aspect, the disclosure provides ingestible compositions comprising the flavor-modifying composition of the second aspect, or any related aspects. In some embodiments, the concentration of the compound of formula (II) in the ingestible composition ranges from 0.1 ppm to 1000 ppm, or from 0.5 ppm to 500 ppm. In some embodiments, the ingestible composition comprises a salty tastant, an umami tastant, a kokumi tastant, a bitter tastant, or any combination thereof.

In a seventeenth aspect, the disclosure provides flavored products comprising the ingestible composition of the sixteenth aspect, or any embodiments thereof. In some embodiments, the flavored products are beverage products, such as soda, flavored water, tea, broth, and the like. In some other embodiments, the flavored products are food products, such as yogurt, soup, and the like. In some embodiments, the flavored product is a meat analogue product or a dairy analogue product. In some embodiments, the flavored product is a seafood analogue product, such as an analogue of fish, shrimp, crab, scallops, and the like. In some embodiments, the flavored product is a meat or seafood product or meat-like product, such as a reformed meat or seafood product, including, but not limited to, ham, sausages, hot dogs, imitation crab meat, kamaboko, and canned meat or seafood products.

Further aspects, and embodiments thereof, are set forth below in the Detailed Description, the Drawings, the Abstract, and the Claims.

### BRIEF DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1 shows a process scheme for the deacetylation process described herein, wherein R¹, R², and R³ are independently a hydrogen atom or —C(O)—CH₃, and at least one of R¹, R², and R³ is —C(O)—CH₃, and R⁴ is C₁₃₋₁₇ alkyl, C₁₃₋₁₇ alkenyl, or C₁₃₋₁₇ alkynyl.

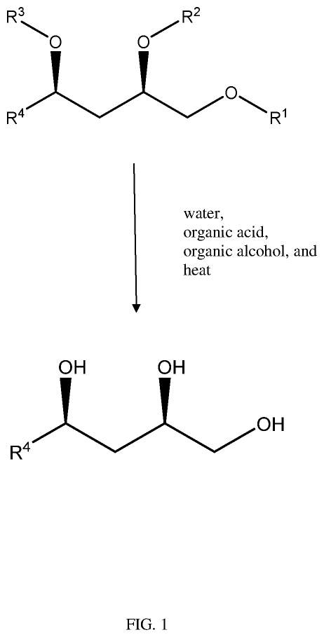

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary As used herein, “Cₐ to C_{b}” or “C_{a-b}” in which “a” and “b” are integers, refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

As used herein, “alkyl” means a straight or branched hydrocarbon chain that is fully saturated (i.e., contains no double or triple bonds). In some embodiments, an alkyl group has 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like. Unless indicated to the contrary, the term “alkyl” refers to a group that is not further substituted. Note that the terms “butyl,” “pentyl,” “hexyl,” and the like refer to straight-chain moieties (and do not encompass branched-chain moieties), unless otherwise indicated to the contrary.

As used herein, “alkenyl” means a straight or branched hydrocarbon chain containing one or more double bonds. In some embodiments, the alkenyl group has from 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₄ alkenyl” or similar designations. By way of example only, “C₂₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like. Unless indicated to the contrary, the term “alkenyl” refers to a group that is not further substituted.

As used herein, “alkynyl” means a straight or branched hydrocarbon chain containing one or more triple bonds. In some embodiments, the alkynyl group has from 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₄ alkynyl” or similar designations. By way of example only, “C₂₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like. Unless indicated to the contrary, the term “alkynyl” refers to a group that is not further substituted.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like.

As used herein, a squiggly bond adjacent to a carbon-carbon double bond indicates that the substituents around the carbon-carbon double bond may connect in either an E or Z configuration, or a combination of molecules in either configuration.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, certain substituents or linking groups having only a single atom may be referred to by the name of the atom. For example, in some cases, the substituent “—H” may be referred to as “hydrogen” or “a hydrogen atom,” the substituent “—F” may be referred to as “fluorine” or “a fluorine atom,” and the linking group “—O—” may be referred to as “oxygen” or “an oxygen atom.”

Points of attachment for groups are generally indicated by a terminal dash (-) or by an asterisk (*). For example, a group such as *—CH₂—CH₃ or —CH₂—CH₃ both represent an ethyl group.

Chemical structures are often shown using the “skeletal” format, such that carbon atoms are not explicitly shown, and hydrogen atoms attached to carbon atoms are omitted entirely. For example, the structure

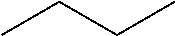

represents butane (i.e., n-butane). Furthermore, aromatic groups, such as benzene, are represented by showing one of the contributing resonance structures. For example, the structure

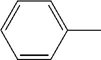

represents toluene.

As used herein a “salty tastant” is a compound that, when ingested, (i) elicits or enhances a perception of salty taste alone or in the presence of a salt, such as sodium chloride or (ii) alters the flow of ions through one or more ion channel associated with perception of salty taste. Examples of ion channels associated with the perception of salty taste include the ENaC channel, the TrpV1 channel and the TrpML3 channel. The term “enhance salty taste” refers to increasing the perception of the presence of a salty tastant, while directly contributing only secondarily (or not at all) to salty taste.

As used herein an “umami tastant” is a compound that, when ingested, (i) elicits or enhances a perception of an umami taste alone or in the presence of another umami tastant or (ii) activates the T1R umami taste receptor. The term “enhance umami taste” refers to increasing the perception of the presence of an umami tastant, while directly contributing only secondarily (or not at all) to umami taste.

As used herein a “bitter tastant” is a compound that, when ingested, (i) elicits or enhances a perception of a bitter taste alone or in the presence of another bitter tastant or (ii) activates one or more T2R bitter taste receptors. The term “mask bitter taste” refers to decreasing the perception of the presence of a bitter tastant.

As used herein a “kokumi tastant” is a compound that, when ingested, elicits or enhances a perception of a kokumi taste alone or in the presence of another kokumi tastant. The kokumi taste is the taste typically associated with gamma-glutamyl tripeptides, fermented foods, and the like. The term “enhance kokumi taste” refers to increasing the perception of the presence of a kokumi tastant, while directly contributing only secondarily (or not at all) to kokumi taste.

As used herein, “juiciness” refers to the presence of liquids, such as water and liquid fat, within a product. The term “enhance perceived juiciness” refers to increasing the perception of the presence of liquid within a product without actually increasing the amount of liquid in the product. For example, compounds that induce increased saliva production can enhance the perception of juiciness.

As used herein, “fattiness” refers to the presence of fatty acid glycerides (for example, triglycerides) within a product. The term “enhance perceived fattiness” refers to increasing the perception of the presence of fatty acid glycerides within a product without actually increasing the amount of fatty acid glycerides in the product.

As used herein, “alcohol” refers to ethanol, when used in the context of describing an ingredient of a food or beverage product.

As used herein, a “pungent tastant” or a “spicy tastant” is a compound that, when ingested, activates the TRpV1 receptor and thereby elicits or enhances a perception of a pungent taste or a spicy taste. Examples of such compounds include capsaicin and the pungent ingredients in mustard and wasabi. The term “reduce pungent taste” or “reduce spicy taste” refers to decreasing the perception of the presence of a pungent tastant or a spicy tastant.

Other terms are defined in other portions of this description, even though not included in this subsection.

### Deacetylation Process

In at least one aspect, the disclosure provides a process for deacetylating acetates, the process comprising:
(a) providing a starting composition comprising one or more avocado acetate compounds of formula (I)

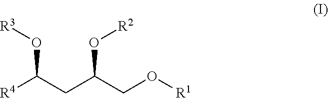

wherein R¹, R², and R³ are independently a hydrogen atom or —C(O)—CH₃, and at least one of R¹, R², and R³ is —C(O)—CH₃, and R⁴ is C₁₃₋₁₇ alkyl, C₁₃₋₁₇ alkenyl, or C₁₃₋₁₇ alkynyl; and
(b) introducing water, an organic acid, and an organic alcohol to the starting composition to hydrolyze the one or more avocado acetate compounds and form a product composition comprising one or more triol compounds of formula (II)

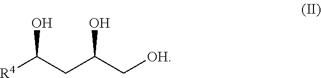

The variables R¹, R², and R³ can have any suitable value, as long as one of them is —C(O)—CH₃. In some embodiments, R¹ and R³ are a hydrogen atom, and R² is —C(O)—CH₃. In some other embodiments, R¹ and R² are a hydrogen atom, and R³ is —C(O)—CH₃. In some other embodiments, R¹ is a hydrogen atom, and R² and R³ are both —C(O)—CH₃. In some other embodiments, all three of R¹, R², and R³ are —C(O)—CH₃.

The variable R⁴ can have any suitable value consistent with the definition set forth above. In some embodiments, R⁴ is C₁₃₋₁₇ alkenyl or C₁₃₋₁₇ alkynyl. In some further embodiments, R⁴ is C₁₃₋₁₇ alkenyl. In some other such embodiments, R⁴ is C₁₃₋₁₇ alkynyl. In some embodiments, R⁴ is —(CH₂)₁₁—CH═CH₂, —(CH₂)₁₁—C—CH, —(CH₂)—CH═CH—(CH₂)8—CH═CH₂, —(CH₂)₁₁—CH═CH—CH═CH₂, —(CH₂)₁₁—CH═CH—CH₂—CH₃, —(CH₂)₁₂—CH₃, —(CH₂)₁₄—CH₃, —(CH₂)₁₇—CH₃, —(CH₂)₁₃—CH═CH₂, —(CH₂)₁₅—CH═CH₂, or —(CH₂)₇—CH═CH—CH₂—CH═CH—(CH₂)₄—CH₃. In some embodiments, R⁴ is —(CH₂)₁₁—CH═CH₂ or —(CH₂)₁₁—C≡CH. In some embodiments, R⁴ is —(CH₂)₁₁—CH═CH₂. In some embodiments, R⁴ is —(CH₂)₁₁—C—CH.

In some embodiments, the avocado acetate compounds of formula (I) comprise acetates of avocadene (acetates of (2R,4R)-1,2,4-trihydroxyheptadec-16-ene) and acetates of avocadyne (acetates of (2R,4R)-1,2,4-trihydroxyheptadec-16-yne). In some such embodiments, the resulting triols of formula (II) comprise avocadene ((2R,4R)-1,2,4-trihydroxyheptadec-16-ene) and avocadyne ((2R,4R)-1,2,4-trihydroxy-heptadec-16-yne).

In a related aspect, the disclosure provides a process for increasing avocado triol content, the process comprising: (a) providing a starting composition comprising avocadene, avocadyne, one or more avocadene acetates, and one or more avocadyne acetates; and (b) introducing water, an organic acid, and an organic alcohol to the starting composition to hydrolyze a portion of the one or more avocadene acetates and a portion of the avocadyne acetates to form avocadene and avocadyne, respectively.

The starting composition can be derived from any suitable source. In some embodiments, the starting composition comprises oil extracted from avocado, for example, the fruit of the Persea americana tree. Such oil can be alternatively referred to as “avocado oil.” In some embodiments, the avocado oil is filtered or otherwise processed to remove residual non-fatty material, such as cellulosic materials. In some embodiments, the starting composition comprises at least 50% by weight, or at least 60% by weight, or at least 70% by weight, or at least 80% by weight, or at least 90% by weight, or at least 95% by weight, or at least 97% by weight, or at least 99% by weight, of avocado oil, based on the total weight of the starting composition.

In some embodiments, the starting composition comprises an amount of triol compounds of formula (II).

In some other embodiments, the starting material comprises an avocado oil fraction.

The fraction may be formed by any suitable means of fractionating seed oils. In some such embodiments, the avocado oil fraction comprises an elevated concentration of one or more compounds of formula (I), according to any of the embodiments set forth above, relative to unfractionated avocado oil. In some such embodiments, the starting composition comprises at least 50% by weight, or at least 60% by weight, or at least 70% by weight, or at least 80% by weight, or at least 90% by weight, or at least 95% by weight, or at least 97% by weight, or at least 99% by weight, of an avocado oil fraction, based on the total weight of the starting composition.

In some embodiments, the starting composition comprises avocado oil and one or more avocado oil fractions. In some such embodiments, the starting composition comprises at least 50% by weight, or at least 60% by weight, or at least 70% by weight, or at least 80% by weight, or at least 90% by weight, or at least 95% by weight, or at least 97% by weight, or at least 99% by weight, of an avocado oil and one or more avocado oil fractions, based on the total weight of the starting composition.

The starting composition comprises one or more avocado acetate compounds of formula (I). In general, the avocado acetate compounds are naturally present in the avocado oil. In some embodiments, the avocado acetate compounds are added to the starting composition, so as to increase the concentration of such compounds in the starting composition compared to their concentration in the avocado oil. The concentration of such compounds may vary from batch to batch for avocado oil.

The deacetylation process disclosed herein comprises introducing water, an organic acid, and an organic alcohol to the starting composition to hydrolyze the one or more avocado acetate compounds and form a product composition comprising one or more triol compounds of formula (II), or any embodiments thereof.

Any form of water can be used in the process. In some embodiments, however, the water is distilled water or deionized water. Any suitable amount of water can be used relative to the amount of avocado oil and/or avocado oil fraction in the starting composition. In some embodiments, the weight ratio of the water introduced to the avocado oil and/or avocado oil fraction ranges from 2:1 to 12:1, or from 3:1 to 10:1.

Any suitable organic acids can be used in the process. In some embodiments, the organic acid is citric acid, oxalic acid, malonic acid, malic acid, tartaric acid, or any combination thereof. In some embodiments, the organic acid is citric acid. Any suitable amount of the organic acid can be used relative to the amount of avocado oil and/or avocado oil fraction in the starting composition. In some embodiments, the weight ratio of the organic acid introduced to the avocado oil and/or avocado oil fraction ranges from 1:3 to 3:1, or from 1:2 to 2:1.

Any suitable organic alcohols can be used in the process. In some embodiments, the organic alcohol is a water-miscible alcohol. In some embodiments, the organic alcohol is methanol, ethanol, isopropanol, or any combination thereof. In some embodiments, the organic alcohol is ethanol. Any suitable amount of the organic alcohol can be used relative to the amount of avocado oil and/or avocado oil fraction in the starting composition. In some embodiments, the weight ratio of the organic alcohol introduced to the avocado oil and/or avocado oil fraction ranges from 3:1 to 20:1, or from 6:1 to 18:1.

The deacetylation reaction is typically carried out with the application of heat for a period of time. In some embodiments, the reaction mixture is heated to a temperature of at least 60° C., or at least 70° C., or at least 80° C., and no greater than 100° C., or no greater than 95° C., or no greater than 90° C. The heat can be applied for any suitable period of time. In some embodiments, the heat is applied for a period ranging from 6 hours to 48 hours, or from 12 hours to 36 hours.

In some embodiments, the heat is applied at a temperature and for a time to cause the evaporation of much of the organic alcohol and a portion of the water to form a concentrated product composition. For example, in some embodiments, at least 90% by weight, or at least 95% by weight, of the organic alcohol is evaporated. In some embodiments, the evaporation results in a two-phase system, with the organic phase containing most, if not nearly all, of the desired triol compounds of formula (II).

In some embodiments, the resulting organic phase can be separated from the aqueous phase of the concentrated product composition and subjected to one or more washings. Such washings can be carried out with water, or with an aqueous base solution, such as an aqueous carbonate solution.

In some embodiments, at least a portion of the optionally washed organic phase of the concentrated product composition is fractionated to isolate a fraction comprising a higher concentration of triol compounds of formula (II), namely an enriched product composition.

The fractionating can be carried out by methods known in the art, such as through crystallization, extraction, chromatography, and the like. In some embodiments, the fractionation is carried out by chromatography using standard organic solvents. The enriched product composition can contain any suitable concentration of compounds of formula (II).

For example, in some embodiments, the enriched product composition comprises at least 20% by weight, or at least 30% by weight, or at least 40% by weight, or at least 50% by weight, of compounds of formula (II), based on the total weight of the enriched product composition.

The process can result in any suitable conversion of the acetate compounds of formula (I) to the triol compounds of formula (II). In some embodiments, at least 30% by weight, or at least 40% by weight, or at least 50% by weight, or at least 60% by weight, or at least 70% by weight, or at least 80% by weight, or at least 90% by weight, of the acetate compounds of formula (I) are converted (and recovered) to triol compounds of formula (II).

In general, the product composition or enriched product composition has a relatively low concentration of avocado acetates of formula (I). For example, in some embodiments, the product composition or enriched product composition comprises no more than 25% by weight, or no more than 20% by weight, or no more than 15% by weight, or no more than 10% by weight, or no more than 5% by weight, of avocado acetates of formula (I).

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Flavor-Modifying Composition Compositions produced by the processes described above have utility as flavor-modifying compositions, for example, for enhancing certain tastes, such as salty taste, umami taste, kokumi taste, and the like.

In certain embodiments, such flavor-modifying compositions comprise one or more triol compounds of formula (II). Such triol compounds can make up any suitable concentration in the flavor-modifying composition. For example, in some embodiments, the flavor-modifying compositions comprise at least 30% by weight, or at least 40% by weight, or at least 50% by weight, or at least 60% by weight, or at least 70% by weight, or at least 80% by weight, or at least 90% by weight, of triol compounds of formula (II).

In some embodiments, the flavor-modifying compositions comprise avocadene ((2R,4R)-1,2,4-trihydroxyheptadec-16-ene) and avocadyne ((2R,4R)-1,2,4-trihydroxy-heptadec-16-yne). In such embodiments, the avocadene and avocadyne can be present in any suitable ratio relative to each other. For example, in some embodiments, the weight ratio of avocadene to avocadyne in the flavor-modifying composition ranges from 1:2 to 5:1, or from 1:1 to 3:1. Avocadene and avocadyne may make up any suitable concentration in the flavor-modifying composition. For example, in some embodiments, the avocadene and avocadyne, collectively, make up at least 10% by weight, or at least 15% by weight, or at least 20% by weight, or at least 25% by weight, or at least 30% by weight, or at least 35% by weight, or at least 40% by weight, of the flavor-modifying composition.

The flavor-modifying compositions can also include other triol compounds of formula (II). Such other triol compounds include, but are not limited to, the following:
(2R,4R)-1,2,4-trihydroxyheptadeca-6,16-diene;
(2R,4R)-1,2,4-trihydroxynonadeca-16,18-diene;
(2R,4R)-1,2,4-trihydroxyheptadecane;
(2R,4R)-1,2,4-trihydroxyheneicosa-12,15-diene;
(2R,4R)-1,2,4-trihydroxynonadecane;
(2R,4R)-1,2,4-trihydroxyheneicosane;
(2R,4R)-1,2,4-trihydroxynonadec-18-ene; and
(2R,4R)-1,2,4-trihydroxyheneicos-20-ene.
(2R,4R)-1,2,4-trihydroxynonadec-16-ene
   Such additional triols can be present in the flavor-modifying composition in any suitable concentration depending, for example, on the natural concentration of their acetates in the starting composition.

### Uses and Methods

In certain aspects, the disclosure provides uses and methods of using the flavor-modifying compositions (in any form according to the preceding aspects and embodiments thereof).

In certain aspects, the disclosure provides uses of the flavor-modifying compositions set forth above, for example in a food or beverage product. In a related aspect, the disclosure provides corresponding methods of using the flavor-modifying compositions set forth above, the method comprising introducing the flavor-modifying composition to an ingestible composition, such as a flavored food or beverage product.

In certain aspects, the disclosure provides uses of the flavor-modifying compositions set forth above to modify a flavor of an ingestible composition. In a related aspect, the disclosure provides corresponding methods of using the flavor-modifying compositions set forth above to modify the flavor of an ingestible composition, the method comprising introducing the flavor-modifying composition to an ingestible composition. In some embodiments, the ingestible composition is a flavored product such as a flavored food or beverage product.

In certain aspects, the disclosure provides uses of the flavor-modifying compositions set forth above to enhance a salty taste of an ingestible composition. In related aspects, the disclosure provides uses of the flavor-modifying compositions set forth above to reduce the salt (e.g., sodium chloride) content of an ingestible composition. In a related aspect, the disclosure provides corresponding methods of using any flavor-modifying compositions set forth above to enhance a salty taste of an ingestible composition, the method comprising introducing the flavor-modifying compositions to an ingestible composition. In some embodiments of these aspects, the ingestible composition comprises a salty tastant, such as sodium chloride, potassium chloride, or any combination thereof. In some embodiments, the ingestible composition is a flavored product such as a flavored food or beverage product. In certain embodiments, the introduction or use of the flavor-modifying compositions permits reduction of the salty tastant (e.g., sodium chloride) by at least 10%, or at least 20%, or at least 30%, or at least 40%, or at least 50%, or at least 60%, or at least 70%, or at least 80%, or at least 90%.

In certain aspects, the disclosure provides uses of the flavor-modifying compositions set forth above to enhance an umami taste of an ingestible composition. In related aspects, the disclosure provides uses of the flavor-modifying composition of the second aspect, or any related aspects, to reduce or eliminate an umami tastant (e.g., glutamate or aspartate) content of an ingestible composition. In related aspects, the disclosure provides corresponding methods of using the flavor-modifying compositions set forth above to enhance an umami taste of an ingestible composition, the method comprising introducing the flavor-modifying composition to an ingestible composition. In some embodiments of these aspects, the ingestible composition is free or substantially free (for example, contains no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm) of monosodium glutamate (MSG). In some embodiments, the ingestible composition is a flavored product such as a flavored food or beverage product.

In certain aspects, the disclosure provides uses of the flavor-modifying compositions set forth above to enhance a kokumi taste of an ingestible composition. In a related aspect, the disclosure provides uses of the flavor-modifying compositions set forth above to reduce or eliminate glutamyl (e.g., L-glutamyl tripeptides) content of an ingestible composition. In another related aspect, the disclosure provides uses of the flavor-modifying compositions set forth above to reduce or eliminate animal (e.g., animal broth or meat) content of an ingestible composition. In a related aspect, the disclosure provides corresponding methods of using the flavor-modifying compositions set forth above to enhance a kokumi taste of an ingestible composition, the method comprising introducing the flavor-modifying composition to an ingestible composition. In some embodiments, the ingestible composition is a flavored product such as a flavored food or beverage product.

In certain aspects, the disclosure provides uses of the flavor-modifying compositions set forth above to mask a bitter taste of an ingestible composition. In a related aspect, the disclosure provides corresponding methods of using the flavor-modifying compositions set forth above to mask a bitter taste of an ingestible composition, the method comprising introducing the flavor-modifying composition to an ingestible composition. In some embodiments, the ingestible composition is a flavored product such as a flavored food or beverage product. In some embodiments, the ingestible composition comprises a bitter tastant, such as potassium chloride.

In certain aspects, the disclosure provides uses of the flavor-modifying compositions set forth above to enhance a perceived sweetness of an ingestible composition. In a related aspect, the disclosure provides corresponding methods of using the flavor-modifying compositions set forth above to enhance a perceived sweetness of an ingestible composition, the method comprising introducing the flavor-modifying composition to an ingestible composition. In some embodiments, the ingestible composition is a flavored product such as a flavored food or beverage product.

In certain aspects, the disclosure provides uses of the flavor-modifying compositions set forth above to reduce a pungent or spicy taste of an ingestible composition. In a related aspect, the disclosure provides corresponding methods of using the flavor-modifying compositions set forth above to reduce a pungent or spicy taste of an ingestible composition, the method comprising introducing the flavor-modifying composition to an ingestible composition. Such reduction occurs through binding to the TRpV1 receptor and reducing its response to compounds that activate the receptor. In some embodiments, the ingestible composition is a flavored product such as a flavored food or beverage product. In some embodiments, the ingestible composition comprises a pungent tastant or a spicy tastant, such as capsaicin and vanilloids in pungent foods, such as mustard, horseradish, and wasabi.

In certain aspects, the disclosure provides uses of the flavor-modifying compositions set forth above to enhance a perceived juiciness of an ingestible composition. In a related aspect, the disclosure provides corresponding methods of using the flavor-modifying compositions set forth above to enhance a perceived juiciness of an ingestible composition, the method comprising introducing the flavor-modifying composition to an ingestible composition. In some embodiments, the ingestible composition is a flavored product such as a flavored food or beverage product.

In certain aspects, the disclosure provides uses of the flavor-modifying compositions set forth above to enhance a perceived fat content of an ingestible composition. In a related aspect, the disclosure provides corresponding methods of using the flavor-modifying compositions set forth above to enhance a perceived fat content of an ingestible composition, the method comprising introducing the flavor-modifying composition to an ingestible composition. In some related aspects, the disclosure provides uses of the flavor-modifying compositions set forth above to reduce the fat content of an ingestible composition. In a related aspect, the disclosure provides methods of reducing the fat content of an ingestible composition, the method comprising introducing the flavor-modifying compositions set forth above to an ingestible composition. In some embodiments, the ingestible composition is a flavored product such as a flavored food or beverage product. In some embodiments, the ingestible composition comprises one or more non-animal-derived fats, such as cocoa butter.

In certain aspects, the disclosure provides uses of the flavor-modifying compositions set forth above to enhance a perceived animal product content of an ingestible composition.

In a related aspect, the disclosure provides corresponding methods of using the flavor-modifying compositions set forth above to enhance a perceived animal product content of an ingestible composition, the method comprising introducing the flavor-modifying composition to an ingestible composition. In some related aspects, the disclosure provides uses of the flavor-modifying compositions set forth above to reduce or eliminate the animal product content of an ingestible composition. In a related aspect, the disclosure provides methods of reducing or eliminating the animal product content of an ingestible composition, the method comprising introducing the flavor-modifying compositions set forth above to an ingestible composition. In some embodiments, the ingestible composition is a flavored product such as a flavored food or beverage product. In some embodiments, the flavored product is a meat analogue product.

In certain aspects, the disclosure provides uses of the flavor-modifying compositions set forth above to enhance a perceived alcohol content of an ingestible composition (for example, among other things, by activating certain heat receptors). In a related aspect, the disclosure provides corresponding methods of using the flavor-modifying compositions set forth above to enhance a perceived alcohol content of an ingestible composition, the method comprising introducing the flavor-modifying composition to an ingestible composition. In some embodiments, the ingestible composition is a flavored product such as a flavored food or beverage product. In some embodiments, the ingestible content is a low-alcohol or zero-alcohol beverage, such as a hard seltzer product.

In certain aspects, the disclosure provides uses of the flavor-modifying compositions set forth above to enhance mouthfeel of an ingestible composition. In a related aspect, the disclosure provides corresponding methods of using the flavor-modifying compositions set forth above to enhance mouthfeel an ingestible composition, the method comprising introducing the flavor-modifying composition to an ingestible composition. In some embodiments, the ingestible composition is a flavored product such as a flavored food or beverage product.

In a certain aspects, the disclosure provides uses of the flavor-modifying compositions set forth above to reduce the perception of an off note of an ingestible composition. In a related aspect, the disclosure provides corresponding methods of using the flavor-modifying compositions set forth above to reduce a perception of an off note an ingestible composition, the method comprising introducing the flavor-modifying composition to an ingestible composition. In some embodiments, the ingestible composition is a flavored product such as a flavored food or beverage product. In some embodiments, the off note is a note characteristic of plant proteins, such as pea proteins, including a cereal note, a cardboard note, a green note, a pungent note, an astringent note, or any combination thereof.

### Ingestible Compositions

The foregoing uses and methods generally involve the use or introduction of the flavor-modifying composition to an ingestible composition having one or more additional components or ingredients. For example, in at least one aspect, the disclosure provides ingestible compositions comprising any flavor-modifying compositions of the foregoing aspects or embodiments thereof.

The ingestible compositions can include the flavor-modifying compositions (according to any of the embodiments set forth above) in any suitable concentration. In some embodiments, the flavor-modifying compositions are present in an amount sufficient to enhance a taste (e.g., enhance an umami, enhance a kokumi, or enhance a salty taste of the ingestible composition. Thus, in some embodiments, the ingestible composition comprises the flavor-modifying compositions in a concentration of no greater than 1000 ppm, or no greater than 900 ppm, or no greater than 800 ppm, or no greater than 700 ppm, or no greater than 600 ppm, or no greater than 500 ppm, or no greater than 400 ppm, or no greater than 300 ppm, or no greater than 200 ppm, or no greater than 150 ppm, or no greater than 100 ppm, or no greater than 50 ppm, or no greater than 40 ppm, or no greater than 30 ppm, or no greater than 20 ppm, or no greater than 10 ppm. In some embodiments, the flavor-modifying composition is present in a minimum amount, such as 0.1 ppm, 0.5 ppm, or 1 ppm. Thus, in some embodiments, the ingestible composition comprises the flavor-modifying composition in a concentration ranging from 1 ppm to 1000 ppm, or 1 ppm to 900 ppm, or 1 ppm to 800 ppm, or 1 ppm to 700 ppm, or 1 ppm to 600 ppm, or 1 ppm to 500 ppm, or 1 ppm to 400 ppm, or 1 ppm to 300 ppm, or 1 ppm to 200 ppm, or from 1 ppm to 150 ppm, or from 1 ppm to 100 ppm, or from 1 ppm to 50 ppm, or from 1 ppm to 40 ppm, or from 1 ppm to 30 ppm, or from 1 ppm to 20 ppm, or from 0.5 ppm to 1000 ppm, or from 0.5 ppm to 900 ppm, or from 0.5 ppm to 800 ppm, or from 0.5 ppm to 700 ppm, or from 0.5 ppm to 600 ppm, or from 0.5 ppm to 500 ppm, or from 0.5 ppm to 400 ppm, or from 0.5 ppm to 300 ppm, or from 0.5 ppm to 200 ppm, or from 0.5 ppm to 150 ppm, or from 0.5 ppm to 100 ppm, or from 0.5 ppm to 50 ppm, or from 0.5 ppm to 40 ppm, or from 0.5 ppm to 30 ppm, or from 0.5 ppm to 20 ppm, or from 0.1 ppm to 1000 ppm, or from 0.1 ppm to 900 ppm, or from 0.1 ppm to 800 ppm, or from 0.1 ppm to 700 ppm, or from 0.1 ppm to 600 ppm, or from 0.1 ppm to 500 ppm, or from 0.1 ppm to 400 ppm, or from 0.1 ppm to 300 ppm, or from 0.1 ppm to 200 ppm, or from 0.1 ppm to 150 ppm, or from 0.1 ppm to 100 ppm, or from 0.1 ppm to 50 ppm, or from 0.1 ppm to 40 ppm, or from 0.1 ppm to 30 ppm, or from 0.1 ppm to 20 ppm.

In some embodiments of any of the foregoing embodiments, the ingestible composition comprises a salty tastant and a flavor-modifying composition (according to any of the embodiments set forth above). In some embodiments, the salty tastant is sodium chloride, potassium chloride, or a combination thereof. In some such embodiments, the salty tastant is sodium chloride. In some other embodiments, the salty tastant is potassium chloride. In some embodiments, the ingestible composition comprises the salty tastant at a lower concentration than an equally salty-tasting composition not comprising the flavor-modifying composition (according to any of the embodiments set forth above). In some such embodiments, the concentration of the salty tastants is more than 10% less, more than 20% less, more than 30% less, more than 40% less, more than 50% less, more than 60% less, or more than 70% less, or more than 80% less, or more than 90% less than the concentration of salty tastants in an equally salty-tasting composition not comprising the flavor-modifying composition (according to any of the embodiments set forth above). In some embodiments, the salty tastant is present in the ingestible composition at a concentration ranging from 0.001 percent by weight to 1.0 percent by weight, or from 0.001 percent by weight to 0.9 percent by weight, or from 0.001 percent by weight to 0.8 percent by weight, or from 0.001 percent by weight to 0.7 percent by weight, or from 0.001 percent by weight to 0.6 percent by weight, or from 0.001 percent by weight to 0.5 percent by weight, or from 0.001 percent by weight to 0.4 percent by weight, or from 0.001 percent by weight to 0.3 percent by weight, or from 0.001 percent by weight to 0.2 percent by weight, or from 0.001 percent by weight to 0.1 percent by weight, or from 0.01 percent by weight to 1.0 percent by weight, or from 0.01 percent by weight to 0.9 percent by weight, or from 0.01 percent by weight to 0.8 percent by weight, or from 0.01 percent by weight to 0.7 percent by weight, or from 0.01 percent by weight to 0.6 percent by weight, or from 0.01 percent by weight to 0.5 percent by weight, or from 0.01 percent by weight to 0.4 percent by weight, or from 0.01 percent by weight to 0.3 percent by weight, or from 0.01 percent by weight to 0.2 percent by weight, or from 0.01 percent by weight to 0.1 percent by weight, based on the total weight of the ingestible composition.

In some embodiments of any of the foregoing embodiments, the ingestible composition comprises an umami tastant and the flavor-modifying composition (according to any of the embodiments set forth above). In some embodiments, the umami tastant is a glutamate, an arginate, a purine ribonucleitide, or a combination thereof. In some such embodiments, the salty tastant is monosodium glutamate (MSG). In some embodiments, the umami tastant is a purine ribonucleotide (for example, inosine monophosphate (IMP), guanosine monophosphate (GMP), hypoxanthine, inosine, or any combination thereof. In some embodiments, the umami tastant is inosine monophosphate (IMP), guanosine monophosphate (GMP), or a combination thereof. In some embodiments, the ingestible composition comprises the umami tastant at a lower concentration than an equally umami-tasting composition not comprising the flavor-modifying composition (according to any of the embodiments set forth above). In some such embodiments, the concentration of the umami tastants is more than 10% less, more than 20% less, more than 30% less, more than 40% less, more than 50% less, more than 60% less, or more than 70% less, or more than 80% less, or more than 90% less than the concentration of umami tastants in an equally umami-tasting composition not comprising the flavor-modifying composition (according to any of the embodiments set forth above). In some embodiments, the umami tastant is present in the ingestible composition at a concentration ranging from 1 ppm to 1000 ppm, or from 1 ppm to 900 ppm, or from 1 ppm to 800 ppm, or from 1 ppm to 700 ppm, or from 1 ppm to 600 ppm, or from 1 ppm to 500 ppm, or from 1 ppm to 400 ppm, or from 1 ppm to 300 ppm, or from 1 ppm to 200 ppm, or from 1 ppm to 100 ppm, or from 1 ppm to 75 ppm, or from 1 ppm to 50 ppm, or from 1 ppm to 25 ppm, or from 1 ppm to 10 ppm, based on the total weight of the ingestible composition. In some embodiments where the umami tastant is monosodium glutamate, the ingestible composition comprises no more than 10 ppm, or no more than 5 ppm, or no more than 1 ppm of monosodium glutamate, based on the total weight of the ingestible composition.

In certain particular embodiments, the ingestible composition comprises fat, such as animal or vegetable fat, and a flavor-modifying composition (according to any of the embodiments set forth above). In some such embodiments, the introduction of the flavor-modifying composition permits one to use less fat (such as more than 10% less, more than 20% less, more than 30% less, more than 40% less, more than 50% less, more than 60% less, or more than 70% less, or more than 80% less, or more than 90% less) and still achieve a level of fatty characteristic of a comparable product that employs a higher concentration of fat but not the flavor-modifying composition. In some related embodiments, the use of the flavor-modifying composition, permits the elimination of fat from the composition. The fat can be any suitable fat, such as a fat derived from an animal or vegetable fat. In some embodiments, the fat is an animal fat, such as milk fat (including fat in various cheeses), beef fat, pork fat, poultry fat, lamb fat, goat fat, fish oil, butter, and the like. In some other embodiments, the fat is a non-animal fat, such as olive oil, canola oil, corn oil, safflower oil, nut oil, peanut oil, cashew oil, soybean oil, palm oil, palm kernel oil, coconut oil, cocoa butter, and nut butters (such as peanut butter, cashew butter, almond butter, hazelnut butter, and the like). In some embodiments, the fat is cocoa butter. The fat can be present in any suitable concentration in the ingestible composition. In some embodiments, the fat concentration in the ingestible composition ranges from 1 percent by weight to 60 percent by weight, or from 1 percent by weight to 50 percent by weight, or from 1 percent by weight to 40 percent by weight, or from 1 percent by weight to 30 percent by weight, or from 1 percent by weight to 20 percent by weight, or from 1 percent by weight to 10 percent by weight.

In some embodiments of any of the foregoing embodiments, the ingestible composition comprises a kokumi tastant and the flavor-modifying composition (according to any of the embodiments set forth above). In some embodiments, the kokumi tastant is a yeast extract, a fermented food product, cheese, garlic or extracts thereof, a gamma-glutamyl-containing polypeptide, a gamma-glutamyl-containing oligopeptide (such as gamma-glutamyl-containing tripeptides); a flavor-modifying composition (such as a cinnamic acid amide or a derivative thereof), a nucleotide, an oligonucleotide, a plant extract, a food extract, or any combination thereof. In some embodiments, the kokumi tastant is a gamma-glutamyl-containing tripeptide. In some embodiments, the ingestible composition comprises the kokumi tastant at a lower concentration than an equally kokumi-tasting composition not comprising the flavor-modifying composition (according to any of the embodiments set forth above). In some such embodiments, the concentration of the kokumi tastants is more than 10% less, more than 20% less, more than 30% less, more than 40% less, more than 50% less, more than 60% less, or more than 70% less, or more than 80% less, or more than 90% less than the concentration of kokumi tastants in an equally kokumi-tasting composition not comprising the flavor-modifying composition (according to any of the embodiments set forth above). In some embodiments, the kokumi tastant is present in the ingestible composition at a concentration ranging from 1 ppm to 1000 ppm, or from 1 ppm to 900 ppm, or from 1 ppm to 800 ppm, or from 1 ppm to 700 ppm, or from 1 ppm to 600 ppm, or from 1 ppm to 500 ppm, or from 1 ppm to 400 ppm, or from 1 ppm to 300 ppm, or from 1 ppm to 200 ppm, or from 1 ppm to 100 ppm, or from 1 ppm to 75 ppm, or from 1 ppm to 50 ppm, or from 1 ppm to 25 ppm, or from 1 ppm to 10 ppm, based on the total weight of the ingestible composition.

In certain particular embodiments, the ingestible composition comprises alcohol, and the flavor-modifying composition (according to any of the embodiments set forth above). In some such embodiments, the introduction of the flavor-modifying composition permits one to use less alcohol (such as more than 10% less, more than 20% less, more than 30% less, more than 40% less, more than 50% less, more than 60% less, or more than 70% less, or more than 80% less, or more than 90% less) and still achieve a level of alcoholic characteristic of a comparable product that employs a higher concentration of alcohol but not the flavor-modifying composition. In some related embodiments, the use of the flavor-modifying composition permits the elimination of alcohol from the composition. The alcohol can have any suitable concentration in the ingestible compositions. In some embodiments, the alcohol concentration ranges from 1 percent by volume to 50 percent by volume, or from 1 percent by volume to 45 percent by volume, or from 1 percent by volume to 40 percent by volume, or from 1 percent by volume to 35 percent by volume, or from 1 percent by volume to 30 percent by volume, or from 1 percent by volume to 25 percent by volume, or from 1 percent by volume to 20 percent by volume, or from 1 percent by volume to 15 percent by volume, or from 1 percent by volume to 10 percent by volume, or from 1 percent by volume to 5 percent by volume, based on the total volume of the ingestible composition. In some embodiments, the ingestible composition comprises no more than 1 percent by volume alcohol, based on the total volume of the ingestible composition. Such ingestible compositions can be in any suitable form. In some embodiments, the ingestible composition is a food product, such as any of those specifically listed below. In other embodiments, the ingestible composition is a beverage product, such as a soda (such as a hard soda), and the like. The alcohol can present in any suitable form, such as alcohol formed from grains, cane sugar, fruits, and the like.

In some embodiments, ingestible compositions comprise a bitter tastant and a flavor-modifying composition (according to any of the embodiments set forth above). Such bitter tastants include, but are not limited to, active pharmaceutical ingredients (APIs), tannins (such as those in coffee, tea, or wine), ginseng, vitamins, minerals, limonin or nomelin (such as found in citrus juices), caffeine, quinine, catechins, polyphenols, potassium chloride, menthol, other commonly used oral care ingredients, cooling agents (such as N-ethyl-N-(thiophen-2-ylmethyl)-2-(p-tolyloxy)acetamide, N-(1H-pyrazol-3-yl)-N-(thiophen-2-ylmethyl)-2-(p-tolyloxy)acetamide, 2-(4-fluorophenoxy)-N-(1H-pyrazol-3-yl)-N-(thiophen-2-ylmethyl)acetamide, 2-(2-hydroxy-4-methylphenoxy)-N-(1H-pyrazol-3-yl)-N-(thiophen-2-ylmethyl)-acetamide, 2-((2,3-dihydro-1H-inden-5-yl)oxy)-N-(1H-pyrazol-3-yl)-N-(thiophen-2-ylmethyl)-acetamide, 2-((2,3-dihydro-1H-inden-5-yl)oxy)-N-(1H-pyrazol-3-yl)-N-(thiazol-5-ylmethyl)-acetamide, and 2-((5-methoxybenzofuran-2-yl)oxy)-N-(1H-pyrazol-3-yl)-N-(thiophen-2-ylmethyl)-acetamide), plant starches or proteins (such as pea protein, soy protein, chickpea protein, or potato protein), algal proteins or starches, fungal proteins or starches, or alcohol, high-intensity sweeteners, such as saccharin, steviol glycosides, mogrosides, aspartame, and the like. The flavor-modifying composition may be used in combination with such bitter tastants in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1,24:1, or 25:1.

In some instances, one may be able to reduce the amount of sweetener in a product by enhancing the umami or kokumi taste. Thus, in some embodiments, the ingestible composition comprises one or more sweeteners according to any of the embodiments set forth below.

Such sweeteners can be present in any suitable amount. For example, in some embodiments, the sweetener is present in a concentration ranging from 0.1% to 12% by weight. In some embodiments, the sweetener is present in an amount from 0.2% to 10% by weight. In some embodiments, the sweetener is present in an amount from 0.3% to 8% by weight. In some embodiments, the sweetener is present in an amount from 0.4% to 6% by weight. In some embodiments, the sweetener is present in an amount from 0.5% to 5% by weight. In some embodiments, the sweetener is present in an amount from 1% to 2% by weight. In some embodiments, the sweetener is present in an amount from 0.1% to 5% by weight. In some embodiments, the sweetener is present in an amount from 0.1% to 4% by weight. In some embodiments, the sweetener is present in an amount from 0.1% to 3% by weight. In some embodiments, the sweetener is present in an amount from 0.1% to 2% by weight. In some embodiments, the sweetener is present in an amount from 0.1% to 1% by weight. In some embodiments, the sweetener is present in an amount from 0.1% to 0.5% by weight. In some embodiments, the sweetener is present in an amount from 0.5% to 10% by weight. In some embodiments, the sweetener is present in an amount from 2% to 8% by weight. In some further embodiments of the embodiments set forth in this paragraph, the sweetener is sucrose, fructose, glucose, xylitol, erythritol, or combinations thereof. The percentages set forth above are weight percent, based on the total weight of the ingestible composition.

In some other embodiments, the sweetener is present in an amount from 10 ppm to 1000 ppm. In some embodiments, the sweetener is present in an amount from 20 ppm to 800 ppm. In some embodiments, the sweetener is present in an amount from 30 ppm to 600 ppm.

In some embodiments, the sweetener is present in an amount from 40 ppm to 500 ppm. In some embodiments, the sweetener is present in an amount from 50 ppm to 400 ppm. In some embodiments, the sweetener is present in an amount from 50 ppm to 300 ppm. In some embodiments, the sweetener is present in an amount from 50 ppm to 200 ppm. In some embodiments, the sweetener is present in an amount from 50 ppm to 150 ppm. In some further embodiments of the embodiments set forth in this paragraph, the sweetener is a steviol glycoside, a mogroside, a derivative of either of the foregoing, such as glycoside derivatives (e.g., glucosylates), or any combination thereof.

The compositions can include any suitable sweeteners or combination of sweeteners. In some embodiments, the sweetener is a common saccharide sweeteners, such as sucrose, fructose, glucose, and sweetener compositions comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose, D-psicose, L-ribose, D-tagatose, L-glucose, L-fucose, L-arbinose, D-turanose, and D-leucrose. In some embodiments, the sweetener is selected from semi-synthetic “sugar alcohol” sweeteners such as erythritol, isomalt, lactitol, mannitol, sorbitol, xylitol, maltodextrin, and the like. In some embodiments, the sweetener is selected from artificial sweeteners such as aspartame, saccharin, acesulfame-K, cyclamate, sucralose, and alitame. In some embodiments, the sweetener is selected from the group consisting of cyclamic acid, mogroside, tagatose, maltose, galactose, mannose, sucrose, fructose, lactose, allulose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A, other sweet Stevia-based glycosides, chemically modified steviol glycosides (such as glucosylated steviol glycosides), mogrosides, chemically modified mogrosides (such as glucosylated mogrosides), carrelame and other guanidine-based sweeteners. In some embodiments, the sweetener is a combination of two or more of the sweeteners set forth in this paragraph. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose.

The sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, Stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In still other embodiments, the sweetener can be a chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by enzymatic transglycosylation reaction catalyzed by various enzymes possessing transglycosylating activity. In some embodiments, the modified sweetener can be substituted or unsubstituted.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a caloric sweetener, such as sucrose, fructose, xylitol, erythritol, or combinations thereof. In some embodiments, the ingestible compositions are free (or, in some embodiments) substantially free of stevia-derived sweeteners, such as steviol glycosides, glucosylated steviol glycosides, or rebaudiosides. For example, in some embodiments, the ingestible compositions are either free of stevia-derived sweeteners or comprise stevia-derived sweeteners in a concentration of no more than 1000 ppm, or no more than 500 ppm, or no more than 200 ppm, or no more than 100 ppm, or no more than 50 ppm, or no more than 20 ppm, or no more than 10 ppm, or no more than 5 ppm, or no more than 3 ppm, or no more than 1 ppm.

The ingestible compositions can, in certain embodiments, comprise any additional ingredients or combination of ingredients as are commonly used in food and beverage products, including, but not limited to:

acids, including, for example citric acid, phosphoric acid, ascorbic acid, sodium acid sulfate, lactic acid, or tartaric acid;

bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);

coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;

preservatives, including, for example sodium benzoate, potassium benzoate, potassium sorbate, sodium metabisulfate, sorbic acid, or benzoic acid;

antioxidants including, for example ascorbic acid, calcium disodium EDTA, alpha tocopherols, mixed tocopherols, rosemary extract, grape seed extract, resveratrol, or sodium hexametaphosphate;

vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;

clouding agents, including, for example ester gun, brominated vegetable oil (BVO), or sucrose acetate isobutyrate (SAIB);

buffers, including, for example sodium citrate, potassium citrate, or salt;

flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or

starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The ingestible compositions can have any suitable pH. In some embodiments, the flavor-modifying compositions enhance the sweetness of a sweetener under a broad range of pH, e.g., from lower pH to neutral pH. The lower and neutral pH includes, but is not limited to, a pH from 1.5 to 9.0, or from 2.5 to 8.5; from 3.0 to 8.0; from 3.5 to 7.5; and from 4.0 to 7. In certain embodiments, compounds as disclosed and described herein, individually or in combination, can enhance the perceived sweetness of a fixed concentration of a sweetener in taste tests at a compound concentration of 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

The ingestible compositions set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness or licorice taste, compounds that enhance saltiness, compounds that enhance a cooling effect, or any combinations of the foregoing.

Thus, in some embodiments, ingestible compositions disclosed herein the flavor-modifying composition is combined with one or more sweetness enhancing compounds.

Such sweetness enhancing compounds include, but are not limited to, 3-((4-amino-2,2-dioxo-1H-benzol[c][1,2,6]thiadiazin-5-yl)oxy)-2,2-dimethyl-N-propyl-propanamide, N-(1-((4-amino-2,2-dioxo-1H-benzo[c][1,2,6]thiadiazin-5-yl)oxy)-2-methyl-propan-2-yl)isonicotinamide, 4-amino-5,6-dimethylthieno[2,3-d]pyrimidin-2(1H)-one, hesperitin dihydrochalcone, hesperitin dihydrochalcone-4‘—O’glucoside, neohesperitin dihydrochalcone, naringenin, naringin, phloretin, glucosylated steviol glycosides, trilobatin, eriodictyol, homoeriodictyol, brazzein, (2R,3R)-3-acetoxy-5,7,4′-trihydroxyflavanone, (2R,3R)-3-acetoxy-5,7,3′-trihydroxy-4′-methoxyflavanone, rubusosides, or compounds such as those set forth in U.S. Pat. Nos. 8,541,421; 8,815,956; 9,834,544; 8,592,592; 8,877,922; 9,000,054; and 9,000,051, as well as U.S. Patent Application Publication No. 2017/0119032.

The flavor-modifying composition may be used in combination with such other sweetness enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1. In some embodiments of any of the preceding embodiments, the flavor-modifying composition is combined with glucosylated steviol glycosides in any of the above ratios. As used herein, the term “glucosylated steviol glycoside” refers to the product of enzymatically glucosylating natural steviol glycoside compounds. The glucosylation generally occurs through a glycosidic bond, such as an a-1,2 bond, an α-1,4 bond, an α-1.6 bond, a β-1,2 bond, a β-1,4 bond, a β-1,6 bond, and so forth. In some embodiments of any of the preceding embodiments, the flavor-modifying composition is combined with 3-((4-amino-2,2-dioxo-1H-benzo[c][1,2,6]thiadiazin-5-yl)oxy)-2,2-dimethyl-N-propyl-propanamide, N-(1-((4-amino-2,2-dioxo-1H-benzo[c][1,2,6]thiadiazin-5-yl)oxy)-2-methyl-propan-2-yl)isonicotinamide, or any combination thereof, in any of the above ratios.

In some further embodiments, ingestible compositions disclosed herein the flavor-modifying composition is combined with one or more other umami or kokumi enhancing compounds. Such umami enhancing compounds include, but are not limited to, naturally derived compounds, such as (E)-3-(3,4-dimethoxyphenyl)-N-(4-methoxyphenethyl)-acrylamide, or synthetic compounds, such as N-(heptan-4-yl)-benzo[d][1,3]dioxole-5-carboxamide, N¹-(2,4-dimethoxybenzyl)-N²-(2-(pyridin-2-yl)ethyl)-oxalamide, alkyl amides, or any other compounds set forth in U.S. Pat. Nos. 8,735,081; 8,124,121; and 8,968,708. The flavor-modifying composition may be used in combination with such umami enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1,20:1,21:1,22:1,23:1, 24:1, or 25:1.

In some further embodiments, ingestible compositions disclosed herein the flavor-modifying composition is combined with one or more cooling enhancing compounds. Such cooling enhancing compounds include, but are not limited to, naturally derived compounds, such as menthol or analogs thereof, or synthetic compounds, such as any compounds set forth in U.S. Pat. Nos. 9,394,287 and 10,421,727. The flavor-modifying composition may be used in combination with such cooling enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1. In some embodiments, the ingestible composition comprises: N-ethyl-N-(thiophen-2-ylmethyl)-2-(p-tolyloxy)acetamide; N-(1H-pyrazol-3-yl)-N-(thiophen-2-ylmethyl)-2-(p-tolyloxy)acetamide; 2-(4-fluorophenoxy)-N-(1H-pyrazol-3-yl)-N-(thiophen-2-ylmethyl)acetamide; 2-(2-hydroxy-4-methylphenoxy)-N-(1H-pyrazol-3-yl)-N-(thiophen-2-ylmethyl)-acetamide; 2-((2,3-dihydro-1H-inden-5-yl)oxy)-N-(1H-pyrazol-3-yl)-N-(thiophen-2-ylmethyl)-acetamide; 2-((2,3-dihydro-1H-inden-5-yl)oxy)-N-(1H-pyrazol-3-yl)-N-(thiazol-5-ylmethyl)-acetamide; 2-((5-methoxybenzofuran-2-yl)oxy)-N-(1H-pyrazol-3-yl)-N-(thiophen-2-ylmethyl)-acetamide, or any combination thereof in any of the aforementioned ratios. In some embodiments, such ingestible compositions comprise menthol or a menthol analogue. In some further embodiments, such ingestible compositions are compositions for use in oral care products, such as toothpaste, mouthwash, whitening compositions, dentifrices, and the like.

In some further embodiments, ingestible compositions disclosed herein the flavor-modifying composition is combined with one or more bitterness blocking compounds. Such bitterness blocking compounds include, but are not limited to 3-(1-((3,5-dimethylisoxazol-4-yl)methyl)-1H-pyrazol-4-yl)-1-(3-hydroxybenzyl)-imidazolidine-2,4-dione, or other compounds set forth in U.S. Pat. Nos. 8,076,491; 8,445,692; and 9,247,759, or in PCT Publication No. WO 2020/033669. The flavor-modifying composition may be used in combination with such bitterness blockers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1,20:1,21:1,22:1,23:1,24:1, or 25:1.

In some further embodiments, ingestible compositions disclosed herein the flavor-modifying composition is combined with one or more sour taste modulating compounds. Some examples of sour-taste modulating compounds include, without limitation, organic acids, such as citric acid, ascorbic acid, malonic acid, lactic acid, acetic acid, and malic acid. The flavor-modifying composition may be used in combination with such sour taste modulating compounds in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1,20:1,21:1,22:1,23:1,24:1, or 25:1.

In some further embodiments, ingestible compositions disclosed herein the flavor-modifying composition is combined with one or more mouthfeel modifying compounds. Such mouthfeel modifying compounds include, but are not limited to, tannins, cellulosic materials, bamboo powder, and the like. The flavor-modifying composition may be used in combination with such mouthfeel enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1,20:1,21:1,22:1,23:1,24:1, or 25:1.

In some further embodiments, ingestible compositions disclosed herein the flavor-modifying composition is combined with one or more flavor masking compounds. Such flavor masking compounds include, but are not limited to, cellulosic materials, materials extracted from fungus, materials extracted from plants, citric acid, carbonic acid (or carbonates), and the like. The flavor-modifying composition may be used in combination with such mouthfeel enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1,20:1,21:1,22:1,23:1,24:1, or 25:1.

In some aspects related to the preceding aspects and embodiments, the disclosure provides uses of the flavor-modifying composition to enhance the flavor of a flavored composition, such as a flavored article. Such flavored compositions can use any suitable flavors, such as fruit flavors, meat flavors, vegetable flavors, and the like. In some embodiments, the flavored composition is a soup or broth, a chip, a beverage, a breakfast cereal, a baked good, a meat analogue product, a meat product (such as a reformed meat product), a seafood analogue product, a seafood product, a dairy product, or a dairy analogue product.

### Flavored Products

In certain aspects, the disclosure provides flavored products comprising any compositions of the preceding aspects or embodiments thereof. In some embodiments, the flavored products are beverage products, such as soda, flavored water, tea, and the like. In some other embodiments, the flavored products are food products, such as soup, meat analogues, dairy analogues, seafood analogues, meat products (such as reformed meat products, including ham, sausages, hot dogs, and canned meat products), seafood products (such as canned seafood products, imitation crab meat, fish cakes, and kamaboko), and dairy products (such as yogurt, protein drinks, and the like).

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In certain embodiments of any aspects and embodiments set forth herein that refer to a flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, the Spreads category, or the Meat category (such as reformed meats, including ham, sausages, canned meats, and the like).

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consomme) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

The Meat Product category generally refers to edible products produced from the muscle or organ tissue of mammals. Examples of such meat products include reformed meat products, such as ham, canned meat products, sausages, hot dogs, and the like.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

In some embodiments, the flavor-modifying composition as disclosed and described herein, individually or in combination, may be provided in a flavoring concentrate formulation, e.g., suitable for subsequent processing to produce a ready-to-use (i.e., ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

The flavored products set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness, compounds that enhance saltiness, compounds that enhance a cooling effect, or any combinations of the foregoing.

In certain embodiments of any aspects and embodiments set forth herein that refer to a sweetening or flavoring concentrate, the sweetening or flavoring concentrate is a non-naturally-occurring product, such as a composition specifically manufactured for the production of a flavored product, such as food or beverage product.

In one embodiment, the flavoring concentrate formulation comprises i) compounds as disclosed and described herein, individually or in combination; ii) a carrier; and iii) optionally at least one adjuvant. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described in this application. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation has the present compound in a concentration that is at least 2 times of the concentration of the compound in a ready-to-use composition. In one embodiment, the flavoring concentrate formulation has the present compound in a concentration that is at least 5 times of the concentration of the compound in a ready-to-use composition. In one embodiment, the flavoring concentrate formulation has the present compound in a concentration that is at least 10 times of the concentration of the compound in a ready-to-use composition. In one embodiment, the flavoring concentrate formulation has the present compound in a concentration that is at least 15 times of the concentration of the compound in a ready-to-use composition. In one embodiment, the flavoring concentrate formulation has the present compound in a concentration that is at least 20 times of the concentration of the compound in a ready-to-use composition. In one embodiment, the flavoring concentrate formulation has the present compound in a concentration that is at least 30 times of the concentration of the compound in a ready-to-use composition. In one embodiment, the flavoring concentrate formulation has the present compound in a concentration that is at least 40 times of the concentration of the compound in a ready-to-use composition. In one embodiment, the flavoring concentrate formulation has the present compound in a concentration that is at least 50 times of the concentration of the compound in a ready-to-use composition. In one embodiment, the flavoring concentrate formulation has the present compound in a concentration that is at least 60 times of the concentration of the compound in a ready-to-use composition. In one embodiment, the flavoring concentrate formulation has the present compound in a concentration that is up to 100 times of the concentration of the compound in a ready-to-use composition.

The sweetening or flavoring concentrates set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness (e.g., eriodictyol, homoeriodictyol, sterubin, and salts or glycoside derivatives thereof, as well as vanillyl lignans, e.g., matairesinol and other compounds set forth in PCT Publication No. WO 2012/146584), compounds that enhance umami (e.g., rubemamine, rubescenamine, (E)-3-(3,4-dimethoxyphenyl)-N-(4-methoxyphenethyl)acrylamide, and the like), compounds that reduce sourness and/or licorice taste, compounds that enhance saltiness, compounds that enhance a cooling effect, or any combinations of the foregoing.

### Packaged Flavoring Compositions

In some further aspects, the disclosure provides a packaged flavoring composition comprising: (a) a flavor-modifying composition (according to any of the embodiments set forth above); and (b) at least one bulking agent.

The packaged flavoring composition may take any suitable form including, but not limited to, an amorphous solid, a crystal, a powder, a tablet, a liquid, a cube, a glace or coating, a granulated product, an encapsulated form abound to or coated on to carriers/particles, wet or dried, or combinations thereof.

The packaged flavoring composition may contain further additives known to those skilled in the art. These additives include but are not limited to bubble forming agents, bulking agents, carriers, fibers, sugar alcohols, oligosaccharides, sugars, high intensity sweeteners, nutritive sweeteners, flavorings, flavor enhancers, flavor stabilizers, acidulants, anti-caking and free-flow agents. Such additives are for example described by H. Mitchell (H. Mitchell, “Sweeteners and Sugar Alternatives in Food Technology”, Blackwell Publishing Ltd, 2006, which is incorporated herein by reference in its entirety). As used herein, the term “flavorings” may include those flavors known to the skilled person, such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture.

Flavors may also provide breath freshening properties, particularly the mint flavors when used in combination with cooling agents. Flavors may also provide breath freshening properties, particularly the mint flavors when used in combination with cooling agents. These flavorings may be used in liquid or solid form and may be used individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C—8 (citrus fruits), aldehyde C—9 (citrus fruits), aldehyde C—12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, and mixtures thereof. These listings of flavorings are merely exemplary and are not meant to limit either the term “flavoring” or the scope of the disclosure generally.

In some embodiments, the flavoring may be employed in either liquid form and/or dried form. When employed in the latter form, suitable drying means such as spray drying the oil may be used. Alternatively, the flavoring may be absorbed onto water soluble materials, such as cellulose, starch, sugar, maltodextrin, gum arabic and so forth or may be encapsulated. The actual techniques for preparing such dried forms are well-known.

In some embodiments, the tabletop sweetener can be made to be similar to brown sugar. In such embodiments, compounds imparting brown notes can be added to the composition to make it taste more similar to brown sugar.

In some embodiments, the flavorings may be used in many distinct physical forms well-known in the art to provide an initial burst of flavor and/or a prolonged sensation of flavor. Without being limited thereto, such physical forms include free forms, such as spray dried, powdered, beaded forms, encapsulated forms, and mixtures thereof.

Suitable bulking agents include, but are not limited to maltodextrin (10 DE, 18 DE, or 5 DE), corn syrup solids (20 or 36 DE), sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, fructooligosaccharides, cellulose and cellulose derivatives, and the like, and mixtures thereof.

Additionally, granulated sugar (sucrose) or other caloric sweeteners such as crystalline fructose, other carbohydrates, or sugar alcohols can be used as a bulking agent due to their provision of good content uniformity without the addition of significant calories.

In one embodiment, the at least one bulking agent may be a bulking agent described in U.S. Pat. No. 8,993,027.

In one embodiment, the at least one bulking agent may be a bulking agent described in U.S. Pat. No. 6,607,771.

In one embodiment, the at least one bulking agent may be a bulking agent described in U.S. Pat. No. 6,932,982.

In some embodiments, the tabletop sweetener composition may further comprise at least one anti-caking agent. As used herein the phrase “anti-caking agent” and “flow agent” refer to any composition which prevents, reduces, inhibits, or suppresses the at least one sweetener from attaching, binding, or contacting to another sweetener molecule.

Alternatively, anti-caking agent may refer to any composition which assists in content uniformity and uniform dissolution. Non-limiting examples of anti-caking agents include cream of tartar, calcium silicate, silicon dioxide, microcrystalline cellulose (Avicel, FMC BioPolymer, Philadelphia, Pa.), and tricalcium phosphate. In one embodiment, the anti-caking agents are present in the tabletop sweetener composition in an amount from about 0.001 to about 3% by weight of the tabletop sweetener composition.

In some embodiments, the sweetener compositions of any of the preceding aspects and embodiments thereof are encapsulated using typical means for encapsulating flavor or fragrance compounds. Non-limiting examples of such technology are set forth in U.S. Patent Application Publication Nos. 2016/0235102, 2019/0082727, 2018/0369777, 2018/0103667, 2016/0346752, 2015/0164117, 2014/0056836, 2012/0027866, 2010/0172945, and 2007/0128234, as well as U.S. Pat. Nos. 7,488,503, 6,416,799, 5,897,897, 5,786,017, 5,603,971, 4,689,235, 4,610,890, 3,704,137, 3,041,180, and 2,809,895. All of the preceding patent publications and patents are hereby incorporated by reference as though set forth herein in their entireties.

### Non-Animal Protein Materials and Products Made Therefrom

Products intended to replace or substitute meat, seafood, or dairy products often rely on various non-animal-based materials, such as fibers and proteins derived from plants, algae, or fungi, to simulate the texture and flavor of meat or dairy. Non-limiting examples of such plant proteins include soy proteins, pea proteins, bean proteins, grain proteins, and the like. Due to compositional differences between such plant-based materials and animal-derived materials, such as a lack of glutamate-containing proteins and glutathione, these products can lack the umami or kokumi taste that consumers traditionally associate with meat or dairy products.

Thus, in certain aspects, the disclosure provides a flavored product comprising a plant-based material (such as a plant-based starch, a plant-based protein, or a combination thereof) and a flavor-modifying composition (according to any aspects and embodiments set forth above). In some further embodiments, the flavored product can include any features of combination of features set forth above for ingestible compositions that contain the flavor-modifying composition. In some embodiments, the flavored product is a beverage, such as soy milk, almond milk, rice milk, oat milk, a protein drink, a meal-replacement drink, or other like product. In some other embodiments, the flavored product is a meat-replacement product, such as a plant-based chicken product (such as a plant-based chicken nugget), a plant-based beef product (such as a plant-based burger), and the like. In some other embodiments, the flavored product is a seafood-replacement product, such as a plant-based seafood product (such as a plant-based shrimp, a plant-based fish fillet, a plant-based scallop, a plant-based lobster, a plant-based crab, and the like). In some other embodiments, the flavored product is a protein powder, a meal-replacement powder, a plant-based creamer for coffee or tea, and the like. In certain further embodiments, any such flavored products contain additional ingredients, and have additional features, as are typically used in the preparation and/or manufacture of such products. For example, such a flavor-modifying composition (according to any of the embodiments set forth above) may be combined with other flavors and taste modifiers, and may even be encapsulated in certain materials, according to known technologies in the relevant art. Suitable concentrations of the flavor-modifying composition are set forth above.

In some further embodiments analogous to the above embodiments, proteins or starches from algal or fungal sources can be used instead of or in combination with plant starches or proteins.

### Non-Meat Protein Materials and Products Made Therefrom

Certain non-meat animal proteins, such as dairy proteins and proteins from bone broth, are commonly used in food products, and are also sold as the primary ingredient in certain protein powders. Such proteins can impart flavors that lack the full umami or kokumi taste that consumers desire. This is especially true for protein isolates, such as protein isolates of whey protein, collagen protein, casein proteins, and the like. Thus, the present disclosure provides ingestible compositions that include non-meat animal proteins and the flavor-modifying composition (according to any aspects and embodiments set forth above). The flavor-modifying composition can be present in any suitable combination, according to the embodiments set forth in the preceding sections of the present disclosure. In some embodiments, the non-meat animal protein is a bone protein, such as a collagen protein derived from the bones of an animal, such as a cow, pig, donkey, horse, chicken, duck, goat, goose, rabbit, lamb, sheep, buffalo, ostrich, camel, and the like. In some embodiments, the non-meat animal protein is a milk protein, such as a whey protein, a casein protein, or any combination thereof. The milk can be the milk of any suitable animal, such as a cow, donkey, horse, sheep, buffalo, camel, and the like.

The flavor-modifying compositions can also be included in certain food or beverage products that include animal milk or materials derived from animal milk. Such products include cheeses, cheese spreads, yogurt, kefir, milk, processed dairy products, cottage cheese, sour cream, butter, and the like.

### Methods of Preparation

The flavor-modifying compositions disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary or desirable to protect sensitive or reactive groups on any of the molecules concerned.

### Examples

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1: Deacetylation Process

15 Kg of avocado oil 911430 (Supplier Expanscience, Epernon, France), 30 kg of 50% w/w citric acid in water and 120 kg of ethanol (Flavor Quality, 96%) were stirred at 85° C. for a total of 48 hours. 75 Kg of water was added and the ethanol was evaporated until two phases were obtained. 15 Kg of organic phase is washed with 10 kg 5% carbonate solution then water and subsequently used for the purification.

2 Kg of the organic phase in 3 kg of cyclohexane were carefully placed on top of a pre-conditioned column containing 6 kg of silica gel. The column was eluted with 25 L of pure cyclohexane (solvent 1), 25 L of cyclohexane:ethyl acetate 4:1 (solvent 2), 25 L of cyclohexane:ethyl acetate 3:2 (solvent 3), 25 L of cyclohexane:ethyl acetate 1:1 (solvent 4), and finally 25 L of pure ethyl acetate (solvent 5) at a flow of 40-55 L/h. Other solvent and ratio could be used for the purification. Fractions of 5-10 L were collected as soon as solvent 4 was charged and their composition was checked by TLC. The fractions containing the products of interest were pooled and the solvent evaporated to dryness. Between 550-650 g of Avocadene (30%) were recovered for an average yield of 30% for the oil and above 90% for avocadene mass balance.

The mixture so obtained was characterized by UPLC/MS. It contained generally 24-32% w/w avocadene and 10-16% avocadyne quantified using an external standard calibration.

### UPLC/MS Method

An Acquity I-Class UPLC system (Waters) including a PDA detector, coupled with a Q-Exactive Plus high-resolution mass spectrometer (Thermo) was used. The column was an Acquity BEH C8 (1.7 μm, 2.1×100 mm) eluted with 0.4 ml/min of H2O/ACN 0.1% formic acid. The gradient was as follows: 50% B, 4.5 min; 50-100% B in 5.5 min; 100% B, 5 min; equilibration for 1 min. The MS was operated in the positive ESI mode, voltage 3500V, probe heater (source) 350° C., capillary 250° C., sheath gas 45, aux gas 10. Full scan ddMS2 mode [100-700 Da] with a resolution of 35000 (FS) and 17500 (ddMS2) was used with stepped NCEs of 10, 30 and 45V and triggering of the MS2 by the top4 most intense ions. The system was run using XCalibur software. Data processing was done using FreeStyle software. Integration of peaks was done using ISIS algorithm on the base peak chromatogram [200-700 Da]. The parameters used were baseline window 80, area noise factor 500 and peak noise factor 10.

### Preparative LC Method (Isolation of Products for NMR)

The system was composed of a Shimadzu LC—20AR high pressures binary pump (up to 20 mL/min), a LH-40 liquid handler injector, fraction collector, a UV/VIS SPD-20A detector and a Sedere ELSD-90-LT detector. The system was run using LabSolutions software. Three columns were used, depending on the products: XBridge BEH C18 OBD Prep Column (130A, 5 μm 10×250 mm); XSelect CSH C18 Prep Sum 10×150 mm; Xselect CSH Prep Fluoro Phenyl column 5 μm 10×100 mm and they were eluted with 7 ml/min of H2O/ACN 0.1% formic acid in gradient mode.

### Example 2—Sensory Testing with Salt Reduction

Sensory testing was carried out using a panel of four (4) expert tasters, who rated the taste of the a mushroom-based cream soup with: (a) a normal amount of salt (0.855%); (b) 20% reduced salt (0.684%); and (c) 20% reduced salt (0.684%) with 1 ppm of the avocadene-avocadyne composition prepared according to Example 1 (C1). The panelists rated the soups for salt intensity, umami intensity, bitter intensity, and overall liking on a scale from 1 to 10. The results are shown in Table 1.

TABLE 1

| | Saltiness | Umami | Bitter | Overall Liking |
|---|---|---|---|---|
| Reg. Salt | 5.8 | 4.8 | 0.0 | 6.3 |
| Red. Salt | 3.0 | 3.0 | 0.3 | 3.3 |
| Red. Salt + C1 | 5.0 | 4.3 | 0.0 | 4.8 |

Further sensory testing was carried out using a panel of three (3) expert tasters, who rated the taste of a meat flavoring formulation with: (a) a normal amount of salt (0.821%); (b) 20% reduced salt (0.646%); and (c) 20% reduced salt (0.646%) with 1 ppm of the avocadene-avocadyne composition prepared according to Example 1 (Cl). The panelists rated the soups for salt intensity, umami intensity, bitter intensity, and overall liking on a scale from 1 to 10. The results are shown in Table 2.

TABLE 2

| | Saltiness | Umami | Bitter | Overall Liking |
|---|---|---|---|---|
| Reg. Salt | 6.0 | 3.3 | 3.0 | 5.0 |
| Red. Salt | 5.7 | 3.3 | 2.3 | 5.3 |
| Red. Salt + C1 | 5.3 | 4.7 | 1.0 | 5.7 |

Sensory testing was carried out using a panel of four (4) expert tasters, who rated the taste of the a green soup with vegetable flavor having: (a) a normal amount of salt (0.6%); (b) 25% reduced salt (0.46%), which also included 0.05% Yeast 444 (Y); and (c) 20% reduced salt (0.46%) with 1 ppm of the avocadene-avocadyne composition prepared according to Example 1 (C1), which also included 0.05% Yeast 444. The panelists rated the soups for salt intensity, umami intensity, bitter intensity, and overall liking on a scale from 1 to 10. The results are shown in Table 3.

TABLE 3

| | Saltiness | Umami | Bitter | Overall Liking |
|---|---|---|---|---|
| Reg. Salt | 5.3 | 4.5 | 0.0 | 4.8 |
| Red. Salt | 4.8 | 4.4 | 0.0 | 5.5 |
| Red. Salt + C1 | 4.4 | 4.9 | 0.0 | 5.3 |

Sensory testing was carried out using a panel of three (3) expert tasters, who rated the taste of the a beef bouillon with no MSG and no salt and no pepper having: (a) a reduced amount of salt (0.38%); (b) a reduced salt (0.38%), KCl (0.4%), (E)-3-(3,4-dimethoxy-phenyl)-N-(4-methoxyphenethyl)acrylamide (E1) (15 ppm); and (c) a reduced salt (0.38%), KCl (0.4%), (E)-3-(3,4-dimethoxyphenyl)-N-(4-methoxyphenethyl)acrylamide (E1) (8 ppm), and 1 ppm of the avocadene-avocadyne composition prepared according to Example 1 (Cl).

The panelists rated the soups for salt intensity, umami intensity, bitter intensity, and overall liking on a scale from 1 to 10. The results are shown in Table 4.

TABLE 4

| | Saltiness | Umami | Bitter | Overall Liking |
|---|---|---|---|---|
| Red. Salt | 4.7 | 4.7 | 1.0 | 6.7 |
| Red. Salt + KCl + E1 | 4.7 | 4.7 | 1.3 | 6.0 |
| Red. Salt + KCl + E1 + C1 | 4.3 | 5.3 | 0.7 | 6.7 |

Sensory testing was carried out using a panel of four (4) expert tasters, who rated the taste of the potato chip having: (a) a normal amount of salt (1.5%); (b) a reduced amount of salt (0.8%), potassium chloride (0.5%), monosodium glutamate (MSG) (0.2%), and ribotide (R1) (0.05%); and (c) a reduced amount of salt (0.8%), potassium chloride (0.5%), monosodium glutamate (0.2%), and ribotide (0.05%), and 1 ppm of the avocadene-avocadyne composition prepared according to Example 1 (C1). The panelists rated the soups for salt intensity, umami intensity, bitter intensity, and overall liking on a scale from 1 to 10. The results are shown in Table 5.

TABLE 5

| | Salti- | | | Overall |
|---|---|---|---|---|
| | ness | Umami | Bitter | Liking |
| Reg. Salt | 5.6 | 3.5 | 1.2 | 5.0 |
| Red. Salt + KCl + MSG + R1 | 6.5 | 4.0 | 1.5 | 6.3 |
| Red. Salt + KCl + MSG + R1 + E1 | 6.5 | 5.0 | 0.8 | 6.3 |

1. A process for deacetylating avocado acetates, the process comprising:
(a) providing a starting composition comprising one or more avocado acetylate compounds of formula (1) 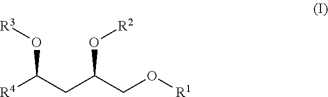
wherein R1, R², and R³ are independently —C(O)—CH₃ or —H, and at least one of R1, R², and R³ is —C(O)—CH₃, and R⁴ is C₁₃₋₁₇ alkyl, C₁₃₋₁₇ alkenyl, or C₁₃₋₁₇ alkynyl; and
(b) introducing water, an organic acid, and an organic alcohol to the starting composition to hydrolyze the one or more avocado acetate compounds and form a product composition comprising one or more triol compounds of formula (II) 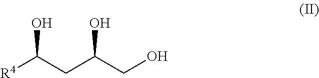
wherein the organic acid is citric acid, oxalic acid, malonic acid, malic acid, tartaric acid, or any combination thereof.

2. The process of claim 1, wherein R¹ and R³ are a hydrogen atom, and R² is —C(O)—CH₃.

3. The process of claim 1, wherein R¹ and R² are a hydrogen atom, and R³ is —C(O)—CH₃.

4. The process of claim 1, wherein R1 is —H, and R² and R³ are —C(O)—CH₃.

5. The process of claim 1, wherein R⁴ is C₁₃₋₁₇ alkyl, C₁₃₋₁₇ alkenyl, or C₁₃₋₁₇ alkynyl.

6. The process of claim 5, wherein R⁴ is

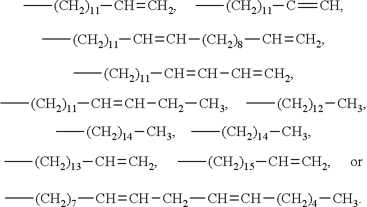

7. The process of claim 6, wherein R⁴ is —(CH₂)₁₁—CH═CH₂ or —(CH₂)₁₁—C═CH.

8. (canceled)

9. The process of claim 1, wherein the organic acid is citric acid.

10. The process of claim 1, wherein the organic alcohol is methanol, ethanol, isopropanol, or any combination thereof.

11. The process of claim 10, wherein the organic alcohol is ethanol.

12. The process of claim 1, further comprising:
(c) removing a portion of the organic alcohol and water from the product composition to form a concentrated product composition having an aqueous phase and an organic phase.

13. The process of claim 12, further comprising:
(d) fractionating of the organic phase of the concentrated product composition to form an enriched product composition comprising at least 20 weight percent triol compounds of formula (II).

14. The process of claim 1, wherein the starting composition comprises avocado oil or avocado oil fraction.

15. A process for increasing avocado triol content, the process comprising:
(a) providing a starting composition comprising avocadene, avocadyne, one or more avocadene acetates, and one or more avocadyne acetates; and
(b) introducing water, an organic acid, and an organic alcohol to the starting composition to hydrolyze a portion of the one or more avocadene acetates and a portion of the avocadyne acetates to form avocadene and avocadyne, respectively, wherein the organic acid is citric acid, oxalic acid, malonic acid, malic acid, tartaric acid, or any combination thereof.

16. The process of claim 15, wherein at least 50% by weight of the avocadene acetates in the starting composition are hydrolyzed to avocadene.

17. The process of claim 15, wherein at least 50% by weight of the avocadyne acetates in the starting composition are hydrolyzed to avocadyne.

18. (canceled)

19. The process of claim 15, wherein the organic acid is citric acid.

20. The process of claim 15, wherein the organic alcohol is methanol, ethanol, isopropanol, or any combination thereof.

21. The process of claim 20, wherein the organic alcohol is ethanol.

22. The process of claim 15, further comprising:
(c) removing a portion of the organic alcohol and water from the product composition to form a concentrated product composition having an aqueous phase and an organic phase.

23. The process of claim 22, further comprising:
(d) fractionating of the organic phase of the concentrated product composition to form an enriched product composition comprising at least 50 weight percent avocadene and avocadyne.

24. The process of claim 23, wherein the concentrated product composition comprises no more than 25 weight percent avocadene acetates and avocadyne acetates.

25. The process of claim 15, wherein the starting composition comprises avocado oil or avocado oil fraction.
